(19) United States
(12) Patent Application Publication
(10) Pub. No.: US 2014/0243783 A1
(43) Pub. Date: Aug. 28, 2014

(54) METHOD OF BACKFLOW REDUCTION DURING MATERIAL DELIVERY THROUGH A NEEDLE INTO TISSUE

(71) Applicants: Raghu Raghavan, Baltimore, MD (US); Martin Brady, Monkton, MD (US)

(72) Inventors: Raghu Raghavan, Baltimore, MD (US); Martin Brady, Monkton, MD (US)

(21) Appl. No.: 14/046,845

(22) Filed: Oct. 4, 2013

Related U.S. Application Data
(60) Provisional application No. 61/710,678, filed on Oct. 6, 2012.

(51) Int. Cl. A61M 25/10; A61M 25/01; A61M 5/158; A61M 25/00

(52) U.S. Cl. CPC A61M 25/10; A61M 25/0023; A61M 25/01; A61M 5/158; A61M 2025/0001; A61M 2210/0693; A61M 2005/1588
USPC 604/506; 604/96.01

(57) ABSTRACT

Interstitial tissue structure is treated with a flowable liquid agent by: providing a delivery cannula having a proximal end and a distal end and an external surface with at least one expandable member located on the external surface of at least one catheter or needle. The delivery cannula is inserted into live tissue, creating a hole in the tissue surrounding the delivery cannula. Contact is made against an inner surface of the hole with contact pressure from the at least one expandable member, said contact pressure establishing a first level of stress in tissue surrounding the hole in the tissue. The expandable member is expanded to increase the contact pressure and provide a second level of stress in the tissue surrounding the hole in the tissue to a second level of stress that is greater than the first level of stress.

### RELATED APPLICATION DATA

This application claims priority under U.S. Law from U.S. Provisional Patent Application Ser. No. 61,710,678, filed Oct. 6, 2012.

### BACKGROUND OF THE INVENTION

1. Field of the Invention

The present invention relates to the field of material delivery into tissue, more particularly the delivery of liquid material, solutions and suspensions such as medication or image enhancing materials into tissue, and material delivery through needles or cannula- or lumen-containing medical elements.

2. Background of the Art

Many liquid and especially aqueous substances are delivered into tissue of patients and even tissue and cavities in the brain or the central nervous system (CNS). Delivery may be chronically or acutely for therapeutic or diagnostic reasons. Delivery of such substances is difficult without introducing backflow (reflux), introducing air, or being able to accommodate variable brain depths with one device.

The introduction of infusion, perfusion and convection-enhanced delivery (CED) has made the CNS amenable to larger delivery volumes, and consequently is a promising approach for treatment of various diseases located in the CNS that respond poorly to systemic chemotherapy or surgical treatment. CED is one of a number of intratis sue delivery mechanisms, including a direct intracranial drug delivery technique that utilizes a bulk-flow mechanism to deliver and distribute macromolecules to clinically significant volumes of solid tissues. This technique shows a greater volume of distribution compared with simple diffusion and is designed to assist in directing a drug to a specific target site. CED bypasses the blood-brain barrier that can be an obstacle for many systemically applied drugs. Studies have been performed to bring CED to clinics. Currently CED is used in clinical trials in patients with recurrent glioblastoma multiforme, Parkinson's and other neurodegenerative diseases.

However, low infusion rate, no standardized catheter design, backflow and reflux are impediments to broad clinical use in CED at the present time. Low infusion rate is associated with long treatment times that expose patients to a higher risk of infections, long periods of physical discomfort and emotional stress. The cannula diameter used in clinics, as well as in previous animal studies, typically allows only a low flow-rate to minimize reflux. It has been shown (Morrison et al.) that in animal experiments that, by reducing diameter of needle (27-32 gauge needle), reflux can be reduced and CED enhanced. However, even a 32 gauge needle, one of the smallest metal needles commercially available, is used with a flow rate of 0.5 μl/min to avoid reflux. Currently there is no standardized cannula design in CED and catheters with large diameters are used in clinical trials. Lidar et al. report that CED led to onset of chemical meningitis during delivery and wound dehiscence after delivery due to leakage of drug in their clinical trial using CED of paclitaxel against malignant gliomas. Thus, side effects caused by reflux of delivered agents may limit safe delivery of therapeutic agent.

U.S. Pat. No. 8,147,480 (Hoofnagle) describes a therapeutic agent delivery system comprising a catheter with a non-bulbous region of an elastically deformable material. In a first state, the bulbous region has a maximum outside diameter greater than substantially uniform outer diameter of the non-bulbous region. While in a second state, the maximum outside diameter of the bulbous regions is reduced relative to the outside diameter when in the first state.

Additional discussion of CED can be found in the following U.S. patents, U.S. published patent applications, and published PCT patent applications, which are incorporated herein by reference in their entirety: U.S. Pat. No. 5,720,720; U.S. Published Patent Application Document Nos. 2009/0143764; 2008/0300571; 2003/0045866; 2003/0045861; 2002/0187127; 2002/0141980; 2002/0114780; and PCT No. WO 0007652.

Clinical application of the convection-enhanced delivery (CED) technique is currently limited by low infusion speed and reflux of delivered agent. Therefore, there is a need for a reflux-resistant cannula which would improve the efficacy of CED. In addition, minimizing the reflux would provide more volume for effective convection and may also allow CED with higher flow rates, which may also improve the therapeutic index of CED and may dramatically reduce the infusion time.

Published U.S. Patent Application Document No. 20070088295 asserts that a step-design cannula and delivery system for chronic delivery of therapeutic substances into the brain using convention-enhanced delivery of therapeutic substances effectively prevents reflux in vivo and maximizes distribution in the brain. That system uses a novel step-design cannula and delivery system for chronic delivery of therapeutic substances into the brain via convection-enhanced delivery (CED). The system overcomes limitations associated with conventional designs used in CED and provides for a reflux-resistant and fast CED method for future clinical trials and asserts a reflux-resistant step-design cannula that allows CED with higher flow rates. The step-design cannula for human use has a tube having a substantially uniform inner diameter (ID). The outer diameter (OD) of the tube, however, is non-uniform and decreases in a step-wise manner from the proximal end to distal end in order to minimize damage in brain tissue and ensure reflux safety. In one beneficial embodiment, the step-design cannula comprises a stainless steel tube having an ID of approximately 0.286 mm (29 gauge) and a length of approximately 234 mm. The OD of the tube is decreases from approximately 5 mm at its proximal end (connection end) to approximately 0.33 mm at its distal end (needle tip) in four steps, thus providing a cannula that has four segments. In this embodiment, the length of the first segment (proximal) is approximately 40 mm with an OD of approximately 5 mm, the length of the second segment is approximately 124 mm with an OD of approximately 2.1 mm, the length of the third segment is approximately 10 mm with an OD of approximately 0.64 mm, and the length of the fourth segment at the distal end (needle tip) is approximately 10 mm with an OD of approximately 0.33 mm. Every change in OD represents a step in the cannula.

All references cited herein are incorporated by reference in its entirety.

### SUMMARY OF THE INVENTION

Interstitial tissue structure is treated with a flowable liquid agent by: providing a delivery cannula having a proximal end and a distal end and an external surface. The cannula carries medical devices and tools such as catheters, needles, syringes, samplers, and the like. There is at least one expandable member located on the external surface of at least one medical device accrued within the delivery cannula, such as a catheter or needle. The delivery cannula is inserted into live tissue, creating a hole in the tissue surrounding the delivery or coring the tissue and perhaps creating a rupture in the tissue surrounding the cannula. Contact is made against an inner surface of the hole with contact pressure from the at least one expandable member, the contact pressure establishing a first level of stress in tissue surrounding the hole in the tissue. The expandable member is expanded to increase the contact pressure and provide a second level of stress in the tissue surrounding the hole in the tissue. The stress level is raised to a second level of stress that is greater than the first level of stress.

### BRIEF DESCRIPTION OF THE FIGURES

FIG. 1 shows a cutaway perspective view of one embodiment of a balloon catheter system within the generic scope of the present technology in a first natural state.

FIG. 2 shows a cutaway perspective view of one embodiment of a balloon catheter system of FIG. 1 within the generic scope of the present technology in an expanded state.

FIG. 3 shows a cutaway perspective view of one embodiment of a balloon catheter system of FIG. 1 within the generic scope of the present technology in an expanded state with a sheath extended that covers the catheter.

FIG. 4 shows a cutaway perspective view of one embodiment of a balloon catheter system within the generic scope of the present technology in an expanded state.

FIG. 5 shows a side view of a balloon covered delivery catheter.

FIG. 6 shows a side view of a balloon covered delivery catheter with a separate lumen for delivery of fluid into the balloon.

FIG. 7 shows a side view of a balloon covered delivery catheter expandable by an internal slideable sheath.

FIG. 8A and FIG. 8B show side views of an expandable balloon catheter in which the balloon is retracted (FIG. 8B from an extended position FIG. 8A).

FIG. 9A and FIG. 9B show side views of an expandable balloon catheter in which the balloon is expanded (FIG. 9B from an extended position FIG. 9A) by release of tension in the extended balloon.

FIG. 10 shows images indicating effects of needle diameters on flow of gel across the surface of the needle.

FIG. 11 shows an image of balloon catheter performance in gel before and after inflation.

### DETAILED DESCRIPTION OF THE INVENTION

In the practice of the present technology, interstitial tissue structure is treated with a flowable liquid agent by: providing a delivery cannula having a proximal end and a distal end and an external surface. The delivery cannula is used for delivery, placement, location or transportation of a medical device or tool for transportation of any flowable mass. The medical device has at least one expandable member located on the external surface of the cannula or the medical device which may comprise at least one catheter or needle. The delivery cannula is inserted into live tissue of a patient (human or non-human animal), creating a hole in the tissue surrounding the delivery cannula. Upon delivery of the medical device, contact is made against an inner surface of the hole with contact pressure from the at least one expandable member on the medical device delivered through or from the delivery catheter creating an initial contact pressure, said initial contact pressure establishing a first level of stress in tissue surrounding the hole in the tissue. The expandable member is expanded to increase the contact pressure between the expandable member and the tissue and to provide a second level of stress in the tissue surrounding the hole in the tissue. The second level of stress that is greater than the first level of stress. The flowable agent (gel or liquid) is delivered into the tissue through the delivery cannula, the expanded expandable member restricting back flow of the liquid agent towards the proximal ends of the needle or catheter. It is particularly important that sheer stress, stress that includes a component that is not perpendicular to the plane of contact between the surface of the needle, cannula, or catheter and the tissue is used in this measurement. The use of elastic piezoelectric sensors (as coating, attachments or inserts) at the interface between the outer surface of the delivery cannula and the tissue is a particularly effective way of sensing and measuring sheer stress. At least one of the contact pressure or the first level of stress are measured, preferably by a sensor on or in the expandable member. The initial contact pressure and/or the first level of stress are measured at a specific point in time to provide a single determinative measurement or else over at least one time period (providing a range of comparable measurements) of at least between one second and at least one minute, which includes being measured over a continuous time period.

When a decrease in contact pressure and/or stress in the surrounding tissue is detected, the expandable member may be expanded an additional amount to increase measured contact pressure and/or stress in the surrounding tissue, if the differential extends outside a tolerable range.

When an increase in contact pressure and/or stress in the surrounding tissue is detected, the expandable member may be deflated an additional amount to decrease measured contact pressure and/or stress in the surrounding tissue, if the differential extends outside a tolerable range.

The delivery cannula may be positioned at or within a treatment region within the tissue of a patient and the delivery cannula is positioned with the distal end within or proximate to said treatment region.

The present technology includes both a medical device and a method of using the medical device. The medical device for controlling flow of delivered flowable material within a patient may have:
a) a cannula for delivering material, smaller devices and material, medical agents, medical compositions and the like (which are deliverable by flow, such as liquids, solutions, suspensions, dispersions, gels, etc.). the cannula having a proximal end and a distal end, and a delivery port towards the distal end of the cannula. The delivery port may be at the extreme end, or merely towards the end, as by side vents or ports;
b) a catheter carrying the cannula, the catheter having an exterior surface and an interior surface;
c) the catheter having a diameter, a distal end and a proximal end, the distal end having an exit port from which the cannula extends or may be extended; and
d) a balloon-element secured to the exterior surface of the catheter between the proximal end and the distal end such that when the balloon-element is expanded, an effectively larger diameter is created about the catheter which can increase force between the secured balloon-element and tissue when the catheter resides within tissue.

The term “balloon-element” is used as opposed to just balloon, as the balloon-element may be inflatable, distendable, expand by longitudinal retraction, expanded by insertion of solid sleeves, and other mechanisms not necessarily associated with traditional fluid expandable balloons, which are within the scope of the broader term, balloon-element. In the device, a source of a fluid selected from the group consisting of gas, liquid and gel may be associated with an interior volume between the balloon-element and the catheter, especially with more traditional balloon style balloon-elements. The source of fluid may be through a tube, hose, pipe, or any other fluid transporting element. It is even possible for a small expandable gas-containing cylinder to be remotely controlled to provide gas into the balloon-element. The device may have the balloon element secured at a proximal end of the balloon-element and a distal end of the balloon element to the exterior surface of the catheter. Where one end (proximal or distal end) of the balloon-element must or should move during expansion of the effective diameter, only one end needs to be secured and at least a portion of the other end must be unsecured to the outside catheter surface. The balloon or balloon-element in most embodiments should be expandable, flexible, stretchable and the like, and both natural (rubber) and synthetic materials (elastomers such as silicone elastomers, polyurethane elastomers, ethylenically unsaturated derived elastomers, fluoroelastomers, etc.) may be used. Typical materials for the structures of the cannula and catheters may also be used, such as polymeric materials, biocompatible metal or metallic materials, composites, ceramics and natural materials.

The method is preferably practiced wherein, in addition to the hole in the tissue from the insertion/penetration of the delivery cannula or other medical element, a tear in the tissue extending from the hole is created due simply to the process of the stress and tearing forces from insertion of the cannula, and where the said method includes an additional step(s) to resist propagation of the tear in the tissue that is performed. Such additional steps may include release of viscous or tacky material into the tear, exudation or release of hardenable material into the tear and hardening of the hardenable material (preferably as it bonds to edges of the tear) as with irradiation, heat, water or sonic activation.

In the practice of these methods, the delivery cannula may be a tubular member having a proximal end and a distal end; said tubular member having a central lumen between said proximal and distal ends; and an infusion tube positioned in said central lumen and extending beyond said proximal and distal ends; said cannula having a plurality of outer surface segments of varying diameter and length; wherein the diameter of each said outer surface segment is substantially uniform along the length of the segment; wherein the relative diameters of said outer surface segments decreases stepwise from the proximal end to the distal end; and wherein the length and diameter of said outer surface segments are selected to reduce reflux during convection-enhanced delivery of substances into tissue in the brain.

Examples of methodology include release of liquid volumes of from 1 μl up to 2000 μl with a flow rate of 0.1, 1.0, 5, 10 and greater than 20 μl/min.

In the use of a step-design cannula in the present invention, the step-design cannula can be inserted into the brain at a desired depth for chronic infusion of agents without introducing air or induction of reflux. Magnetic resonance imaging can be performed with this device to assure correct placement and performance of the device. Conventional devices used for brain delivery are not designed for delivery without introduction of air into the tissue or without reflux, both of which reduce the efficacy of CED. The step design of the cannula has the further advantage of significantly reducing reflux and providing for efficient tissue distribution. Prior art step-design cannulas have sharp, essentially right angle transitions from the surface segment, up to the rising wall segment, and again from the rising wall segment in transit to the next surface segment. Although these sharp and right angle transitions may have some benefit in reducing flow back or reflux, those sharp feature have been found by the inventor to also tend to increase tearing of tissue during insertion and progression of the step-design catheter through the tissue. It has been found that that beveling or rounding of the edges or lines between the transitions (e.g., extending at least 2% up to 25% of the rising wall section(s)) can be expected to reduce tearing with minimum adverse effects on reduction of backflow, with or without the expandable member.

There are transitions between segments in the delivery cannula that may be a wall between cannula surfaces of the delivery cannula parallel to the central lumen and a wall surface approximately perpendicular (e.g., between 45 degrees and perpendicular to the cannula surface) to the central lumen, wherein at least some intersections between the cannula surfaces and the wall surfaces are beveled or rounded.

The delivery cannula may comprise polymeric materials, composite materials, metal and combinations thereof.

It is desirable that any reflux-resistant cannula for chronic delivery of substances to the brain using CED have a tubular member having a proximal end and a distal end; said tubular member having a central lumen between said proximal and distal ends; and an infusion tube positioned in said central lumen and extending beyond said proximal and distal ends; said cannula may have (or may not have) a plurality of outer surface segments of varying diameter and length; wherein the diameter of each said outer surface segment is substantially uniform along the length of the segment. The relative diameters of said outer surface may have segments that decrease stepwise from the proximal end to the distal end; and wherein the length and diameter of said outer surface segments are selected to reduce reflux during convection-enhanced delivery of substances into tissue in the brain.

In another embodiment, a reflux-resistant cannula for chronic delivery of substances to the brain via CED comprises a tubular member having a proximal end and a distal end; said tubular member having a central lumen between said proximal and distal ends; and an infusion tube positioned in said central lumen and extending beyond said proximal and distal ends; said cannula having an outer surface with an inflatable or expandable element (e.g., balloon) that can be inflated after insertion of the cannula into tissue. There may be a single inflatable component or the component may be individual inflatable or expandable components distributed over individual ones of the plurality of stepped segments of varying diameter and length; wherein the diameter of each said stepped segment is substantially uniform along the length of the segment; wherein the relative diameters of said segments decreases stepwise from the proximal end to the distal end; and wherein the length and diameter of said stepped segments are selected to reduce reflux during convection-enhanced delivery of substances into tissue in the brain. The dimensions of the individual inflatable/expandable segments may differ along the length of the steps of the cannula to approximate a consistent diameter along the length of the cannula over the segments so that upon inflation of the individual balloon segments, and approximately uniform diameter (outside the balloons) along the length of the cannula, with little differentiation in outer diameters over each segment.

A reflux-resistant cannula for chronic convection-enhanced delivery of substances to the brain may have a tubular member having a proximal end and a distal end; said tubular member having a central lumen between said proximal and distal ends; and an infusion tube positioned in said central lumen and extending beyond said proximal and distal ends; said cannula having an outer surface with a plurality of stepped segments of varying diameter and length; wherein the diameter of each said stepped segment is substantially uniform along the length of the segment; wherein the relative diameters of said segments decreases stepwise from the proximal end to the distal end; wherein said stepped segments comprise a first segment having a length of approximately 40 mm and an outer diameter of approximately 5 mm, a second segment having a length of approximately 124 mm and an outer diameter of approximately 2.1 mm, a third segment having a length of approximately 10 mm and an outer diameter of approximately 0.64 mm, and a fourth segment having a length of approximately 10 mm and an outer diameter of approximately 0.33 mm; and wherein the length and diameter of said stepped segments are selected to reduce reflux during convection-enhanced delivery of substances into tissue in the brain. Beveled or rounded transitions would be preferred in the construction.

In another embodiment, any of the cannula configurations described above includes a central lumen with a substantially uniform inner diameter. In a still further embodiment, at least a portion of the infusion tube is rigid and at least a portion of the infusion tube is non-rigid.

In another embodiment a system for reflux-resistant chronic convection-enhanced delivery of substances to the brain comprises any of the cannula configurations as described above in combination a tubular delivery sheath having an outer surface, a proximal end and a distal end; said delivery sheath having a central lumen extending from said distal end toward said proximal end; said delivery sheath having a longitudinal passageway which communicates between said central lumen and said outer surface; wherein said passageway is configured for insertion of said cannula into said delivery sheath and through said central lumen. In embodiment, the delivery sheath includes a plurality of openings adjacent said passageway configured for receiving sutures.

In one embodiment of the cannula or system, the infusion tube comprises fused silica. In another embodiment, the tubular member is non-rigid. In another embodiment, the infusion tube is non-rigid. In one embodiment of the system, the delivery sheath is non-rigid.

An expandable injection system useful in the treatment of an interstitial tissue structure with at least one active agent may have, in combination, a syringe or pump system, at least one inflatable member, and a needle or catheter constructed and arranged for interstitially contacting, manually or automatically, precisely measured amounts of said at least one active agent directly into a body tumor or tissue positioned at or within a treatment region within the solid tissue of a patient, while minimizing or preventing backflow of said active agent, and applying compression to said tissue structure to minimize or prevent drainage of said active agent. Sensors may be present on or in the inflatable member.

The compression fluid delivery device includes a catheter member with a proximal end and a distal end and an inner lumen extending therethrough, and dilation/inflatable members preferably disposed proximate to distal end of the catheter or delivery cannula. At least one dilation member can be disposed proximate to the distal end of the catheter. At least a single dilation member catheter must include means for occlusion of the instrument entry track into tissue. The catheter is comprised of a shaft with an inner lumen assembly generally referred to as comprising adjacent multiple lumens. The sensors may be disposed on, under or in the inflatable members. The sensors may communicate by wired or wireless formats to a receiver associated with a processor to evaluate the signals. The shaft of the catheter member and/or the dilation member inner and/or outer wall may be optionally equipped with sensing members electrically coupled to a data logger (not represented) and a controller and monitoring system (not represented).

The catheter/cannula has control means (inflation/deflation, injection) at the proximal end. The distal end of the catheter may be steerable by conventional means. Alternatively, the distal end may be of a flexibility, toughness, and bendability, different from that of shaft. The tip end is preferably closed, blunt or tapered or round, but may be of any desirable shape, flexibility and openness like pointed, sharp, cutting, fully open, pre-bent, or bendable, and/or steerable. Such metal as stainless steel, or metallic alloy, or memory material like nitinol may be used for its structure. Any desirable combination of toughness, slidability, or flexibility and other desirable characteristics for the proximal end and distal end of catheter is within the spirit of the invention.

The outer diameter of the shaft could range in size, for example, from 0.1 Or 0.3 mm for microprocedures, to 2.5 mm or larger for endoscopic procedures, to 10 mm or more for open surgery procedure or direct procedures through natural openings or surgical cavities. The length of the shaft depending on the location of target tissue from the body surface could be in the range of, for example, 15 cm for easily percutaneously accessible tissue such as the prostate gland to 130 cm or up to 200 cm for endoscopic procedure to distal lesion of the arterial, aerial or digestive tract. In the illustrative embodiment, i.e., the apparatus for treating endobronchial tumor through a fiberscope, outside diameter of shaft is 1.67 mm (0.066 inch), and outside diameter of sliding outer sheath is 2.7 mm (0.105 inch), maximal protrusion of distal end out of outer sheath is 30 mm (5 mm to 70 mm), distal end diameter is 16 G (14 G to 21 G) and minimum channel size of endoscope must be 2.8 mm. Usable length of catheter, i.e. from distal end to proximal end of the sheath, may be 1700 mm. Proximal end may be provided with a depth of penetration control means that is made of a sliding stem with a central lumen which has the same pattern of subdivision lumens. Sliding stem may be a thicker and rigid wall of catheter proximal parts whose advancement into outer rigid barrel is controlled by mechanical pressure exerted by a barrel on a stem. Stem bears marking that show depth of protrusion of distal end tip of the catheter out of distal part of outer sheath. Means for depth of insertion control are by no means limited to the system and the advancement and penetration depth of a simple catheter without outer sheath could be controlled with a haemostatic valve located at the proximal entry of the biopsy (operative) channel of an endoscope, provided that during immersion into target tissue the head of the delivery cannula remains stable. Significant movement of the distal end of the cannula would adversely impact both sensing and performance. It is therefore desirable for the sensors to be miniaturized, to have distal communication capability (wired or wireless) and to not require movement during execution of its sensing function.

### Sensors

### Application: Human Interface

Only recently has it become possible to measure shear forces directly at the interface point of two mating surfaces. A shear sensor is an ideal research tool for human body interfaces of clothing, shoes, gloves, backpacks or any object that comes in contact with the human skin and creates shear forces.

Other piezoelectric sensors would have elastomeric binding compositions with distributed conductive fibers or particles distributed therein, and a current flow or voltage across the sensor. Changes in the voltage or current are detected and evaluated as corresponding to changes in dimensions, stress and other physical alterations in the composition of the sensor.

Tactilus® sensor technology reveals the vector of the shearing action in real time. Tactilus® sensor technology can be a valuable aid in human factors engineering and ergonomics, in both research as well as a clinical assessment tool. Computer modeling with FEA software or other analysis tools yielded predictions. With this technology, an engineer can place a sensor at the interface surface and collect shear data in real time.

### Tactilus Technology

Tactilus® sensors are a matrix based tactile surface sensor that works by the principle of piezoresistance. Each Tactilus® sensor is carefully assembled to exacting tolerances and individually calibrated and serialized. The architectural philosophy of Tactilus® is modular allowing for portability, and easy expansion. Tactilus® employs sophisticated mathematical algorithms that intelligently separate signal from noise, and advanced electronic shielding techniques to maximize environmental immunity to noise, temperature and humidity.

The System may include a Shear sensor element, Electronic controller and Software and cables.

### Specifications

| Technology | Piezoresistive |
|---|---|
| Total Sensing Area | 2″ × 2″ (5.1 × 5.1 cm) |
| Force Range | 0-1.5 Newtons |
| Pressure Range | 0-3.87 PSI (0-200 mmHg) |
| Grid Size | 4 × 4 |
| Discrete Sensing Points | 16 |
| Scan Speed | 30 hertz |
| Thickness | 23.6 mils (0.6 mm) |
| Accuracy | ±10% |
| Repeatability | ±2% |
| Hysteresis | ±5% |

### Non-Linearity

Additional elements and functional capacity of the pre-stress catheter system. Each of these aspects of the technology may be used separately, or in some cases, in combination with other aspects of the present novel technology.

An Air-filled elastomeric balloon. The balloon used in the pre-stress technology may extend from a few millimeters to a few centimeters along the catheter shaft (e.g., from 2 mm to 6 cm). The balloon may be expanded from a gas source either by applying a fixed pressure, through a regulator from a controlled pressure (e.g., medium to high pressure) air tank, or by injecting part or all of a fixed volume of air through a syringe.

A Fluid-filled elastomeric balloon. The balloon function is similar to that of the air-filled version, but the balloon is instead inflated with a fluid such a sterile saline solution or biologically safe fluid (e.g., biologically inert synthetic liquids, such as perfluorinated hydrocarbon artificial blood or silicone fluids). A fixed volume (via syringe and stopcock), controlled fluid pressure or fixed fluid pressure may be applied through a lumen or catheter or tube to the balloon.

Elastomeric sheath expanded by way of a solid catheter sleeve. A balloon membrane may cover and conform to the catheter shaft to within a few millimeters of the distal tip, leaving a gap between the shaft and an inner (towards the shaft) surface of the balloon membrane. A solid sleeve would then be placed over the catheter shaft. The solid sleeve has an inside diameter (ID) slightly larger than the catheter (outside diameter (OD), and should be able to slide freely along the shaft. The sleeve fits in the gap between the catheter shaft and the elastomeric sheath. After catheter insertion (without the sleeve), the sleeve is inserted and then slid down to the distal end of the catheter, thus displacing the balloon outward, applying a fixed expansion radius. The sleeve can be made in different wall thicknesses in order to allow for selectable amounts of expansion. Additional control over the expansion radius can be provided by a varying diameter sleeve or some fluid (gas or liquid) expansion capacity in the balloon membrane.

Elastomeric sheath expanded by way of self-expanding stent. Plastic or bio-inert metal (e.g., nitinol) self-expanding stents are normally deployed within a catheter guide tube. In this embodiment, the stent and guide tube would be positioned or fit in coaxial alignment around the main catheter shaft, underneath an elastomeric sheath. The catheter-stent-guide-sheath assembly is inserted first, and then the guide tube is withdrawn. Withdrawal of the guide tube allows the stent to expand, displacing the balloon outward.

Elastomeric sheath expanded by lateral compression. A thin band of elastomeric material (preferably near the tip of the catheter) is compressed longitudinally along the catheter, reducing its length along the catheter and causing the material to effectively expand the band of elastomeric material radially. The compression can be accomplished using a solid catheter sleeve that slides along the catheter shaft or by a retraction component (e.g., pull wire or shaft) that pulls the distal end of the elastomeric material to retract and bunch up to cause effective expansion. Rather than sliding under the elastomer as in the previous embodiment, it pushes on the proximal edge of the elastomer from the proximal side or pulls on the distal edge from the distal side of the elastomeric material.

Elastomeric sheath expanded by release of lateral expansion. In this variant of the compression sheath, the sheath is stretched laterally along the catheter shaft (and retained in the stretched position during placement), narrowing the elastomeric material radially. The catheter is inserted in this stretched condition, and then the sheath is released from the proximal end (or the distal end), shortening the elastomeric element axially (lengthwise) and thickening it radially.

Infused viscoelastic sealant material. The catheter shaft has a coaxial design in which the intended treatment is infused down the center lumen and out from the distal tip. The outer coaxial lumen is used to infuse a biocompatible thick gel sealant material. Radial ports for egress of the gel sealant are placed a distance of at least several millimeters back from the catheter distal tip and may be distributed radially and longitudinally towards the distal tip or the middle of the catheter. After insertion, a small amount of the gel material is infused, forming a seal around the catheter shaft and displacing some of the tissue at that location. The gel may be sufficiently coherent that it may be retracted into the catheter, back through the ports.

An underlying appreciation of the present technology may be appreciated from a review of the Figures. The generic concept includes at least any insertable device, especially material delivery devices, that include an after-insertion, inflatable/expandable cover over the insertable device at a location on the insertable device between the device and penetrated tissues surrounding the inserted device. The balloon is a system that adjusts the pressure between the devioce and the penetrated tissue to restrict fluid flow of materials that have ben delivered at a location (distal or proximal to) the balloon/tissue interface. The inflation of the balloon, even where not interfaced with the tissue, assists in controlling the shape and direction of delivered material movement from the spot of delivery and even along the delivery device. By using the system, methods are provided that allow control of movement of delivered liquid materials or dissolvable materials in ways that can assist medical practitioners in assuring that operation parameters of procedures (e.g., dwell times, persistence of concentrations, rate of movement away from the delivery site, and the like) can be effectively administered.

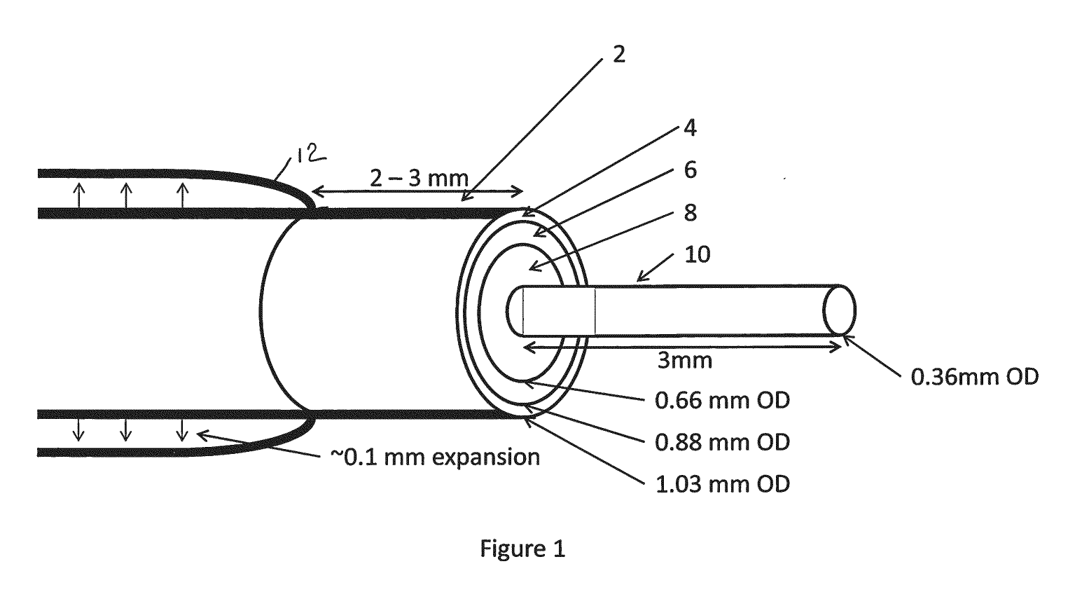

FIG. 1 shows a cutaway perspective view of one embodiment of a balloon catheter system 2 within the generic scope of the present technology in a first natural state. The system 2 comprises a central lumen 10 of small diameter through which fluid carrying the therapeutic flows. The balloon 12 is affixed onto the outer catheter wall acting as a step 6; this may need a thin layer 4 of different material. The embodiment shows a step catheter, the step 6 over the cannula (towards the distal tip) 10 already providing a measure of backflow reduction as would occur in systems evidenced by prior art. The balloon shown here may also be used without the step. A slideable sheath 8 may be used to slide over and protect the elongate element (cannula with a distal tip) or medical device 10. The balloon 12 surrounding the system 2 is shown in a relaxed, loose, partially or naturally inflated state, identified as having a 0.1 mm expansion. All dimensions shown in these figures are for general information and are not limits on the scope of practice within the present invention. The balloon 12 need not be expanded away from the outer catheter wall 4 at all, and may often be provided in a smooth contiguous relationship with that wall 4.

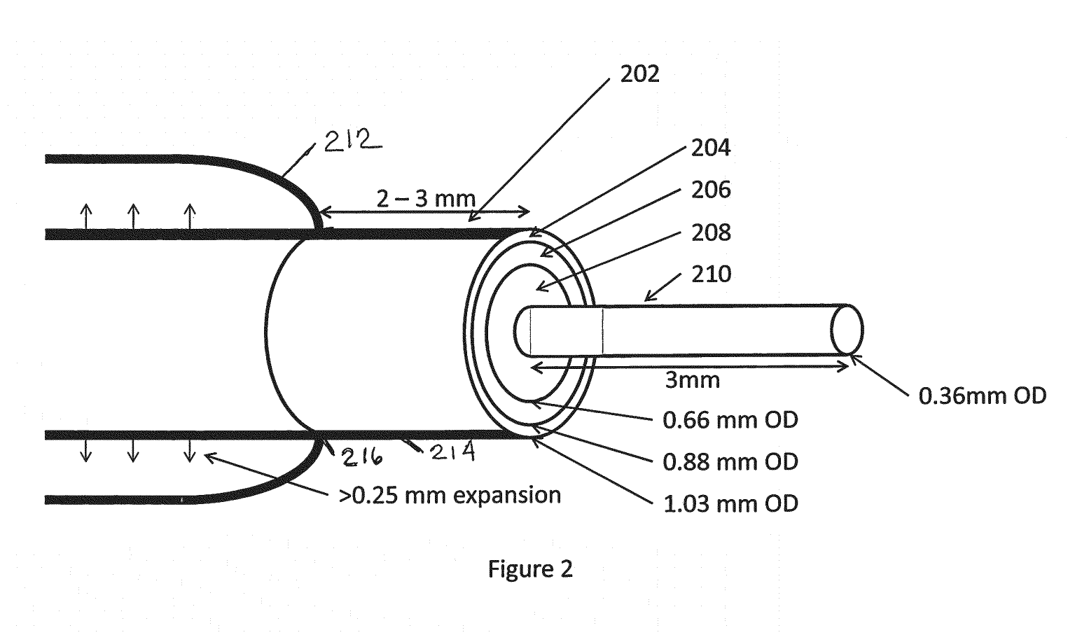

FIG. 2 shows a cutaway perspective view of one embodiment of a balloon catheter system 202 of FIG. 1 within the generic scope of the present technology in an expanded state. The system 202 comprises an outer catheter wall 204, an open fluid transport area 206, a slideable sheath 208 that slides over and protects the central cannula or distal tip 210 which might also carry or be a medical device or delivery device (not shown). A balloon 212 surrounding the system 202 is shown in an inflated state, identified as having a 0.25 mm expansion as opposed to the more relaxed 0.1 mm expansion of FIG. 1. The expanded balloon section 212 may be part of a continuous elastomeric layer 214 with a dividing point 216 where adhesion of the balloon 212 to the outer catheter wall 204 stops, allowing the balloon 212 to be expandable, while the section 214 is not expandable.

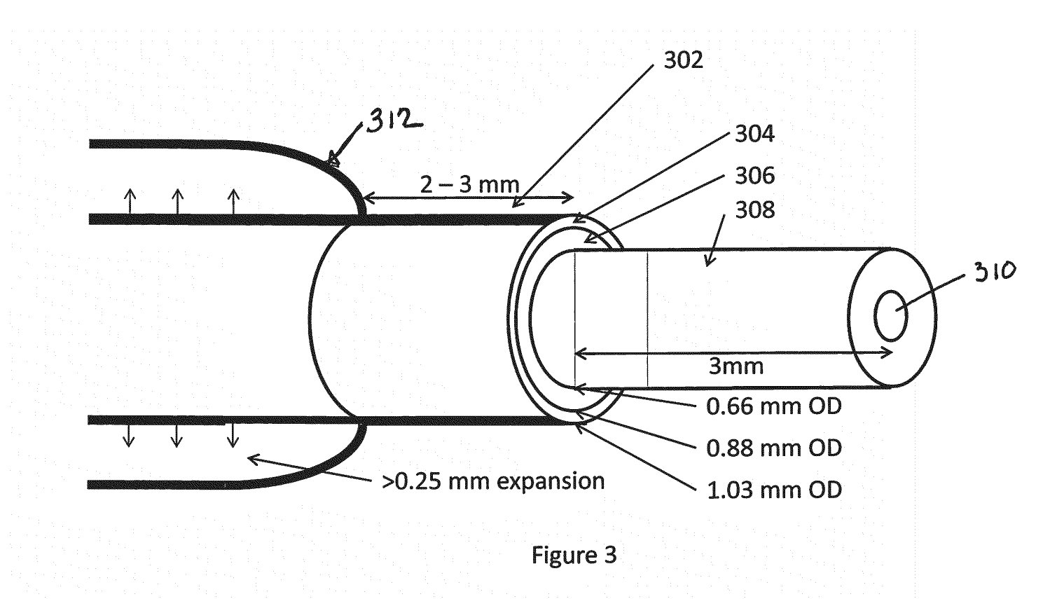

FIG. 3 shows a cutaway perspective view of one embodiment of a balloon catheter system 302 of FIG. 1 within the generic scope of the present technology in an expanded state with a sheath 308 extended so that it covers the catheter. The system 302 comprises an outer catheter wall 304, an open fluid transport area 306, a slideable sheath 308 that has been extended distally along with the elongate element medical device or cannula (which may have a lumen) 310. A balloon 312 surrounding the system 302 is shown in an inflated state, identified as having a 0.25 mm expansion as opposed to the more relaxed 0.1 mm expansion of FIG. 1.

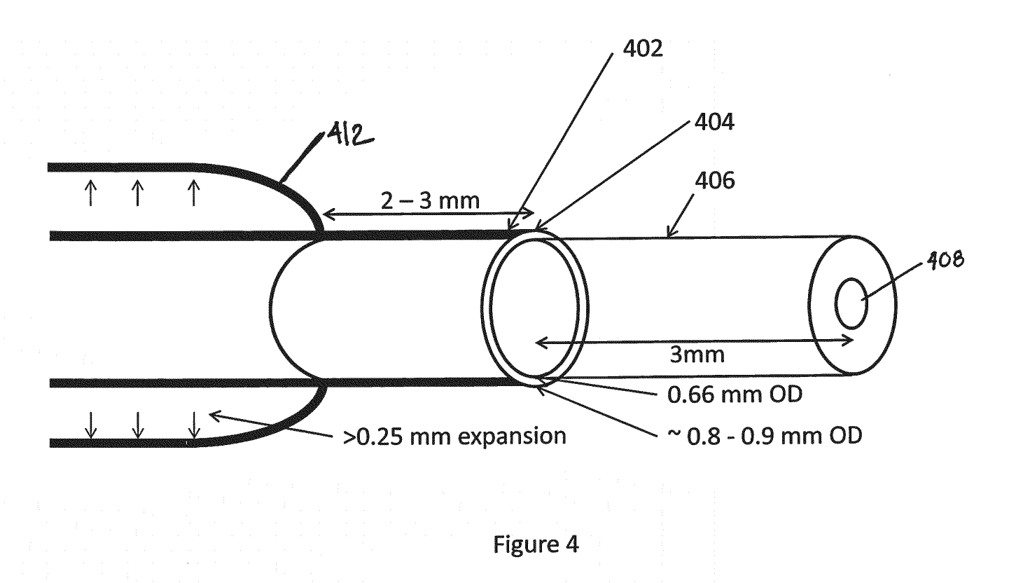

FIG. 4 shows a cutaway perspective view of one embodiment of a balloon catheter system 402 within the generic scope of the present technology in an expanded state. In this embodiment, there is a lumen 408 through which the drug-carrying liquid flows. The lumen is contained within a tube cannula 406 that is carried within the catheter 402. The balloon has been inflated by either a distal fluid source (not shown) or from internal catheter sources (not shown). The cannula 408 within the catheter 406 is also shown, as is the balloon 412 in an inflated state,

The pre-stress catheter system has additional embodiments described below. Each of these aspects of the technology may be used separately, or in some cases, in combination with other aspects of the present novel technology.

An Air-filled elastomeric balloon. The balloon used in the pre-stress technology may extend from a few millimeters to a few centimeters along the catheter shaft (e.g., from 2 mm to 6 cm). The balloon may be expanded from a gas source either by applying a fixed pressure, through a regulator from a controlled pressure (e.g., medium to high pressure) air tank, or by injecting part or all of a fixed volume of air through a syringe. FIG. 5 shows an air-filled elastomeric system 500, with the balloon 502, the proximal end of the catheter 504, the distal end of the catheter 506, the internal (to the balloon) section of the catheter 508 and a port 510 in the internal (to the balloon) section of the catheter 508 from which air (or other fluid) may be delivered to create a volume 512 separating the balloon 502 from the internal (to the balloon) section of the catheter 508 to create stress and an expanded area. Lumen 514 in the catheter 504 is used to deliver fluid (gas or liquid) into the balloon 502 to create expanded volume 512. A central lumen 516 can be used to carry liquid to be delivered and/or a medical device.

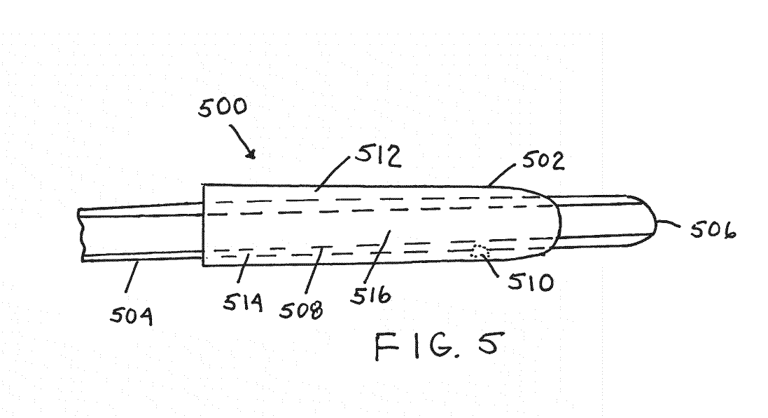

A Fluid-filled elastomeric balloon. This design might look identical to the air filled elastomeric balloon system 500 of FIG. 5, although variations are available. The balloon function is similar to that of the air-filled version, but the balloon is instead inflated with a fluid such a sterile saline solution or biologically safe fluid (e.g., biologically inert synthetic liquids, such as perfluorinated hydrocarbon artificial blood or silicone fluids). A fixed volume (via syringe and stopcock), controlled fluid pressure or fixed fluid pressure may be applied through a lumen or catheter or tube to the balloon. FIG. 6 shows a fluid-filled elastomeric balloon system 600, with the balloon 602, the proximal end of the catheter 604, the distal end of the catheter 606, the internal (to the balloon) section of the catheter 608 and a fluid-carrying tube 614 in communication with the internal (to the balloon) section of the catheter 608 from which fluid may be delivered to create a volume 612 separating the balloon 602 from the internal (to the balloon) section of the catheter 608 to create stress and an expanded area.

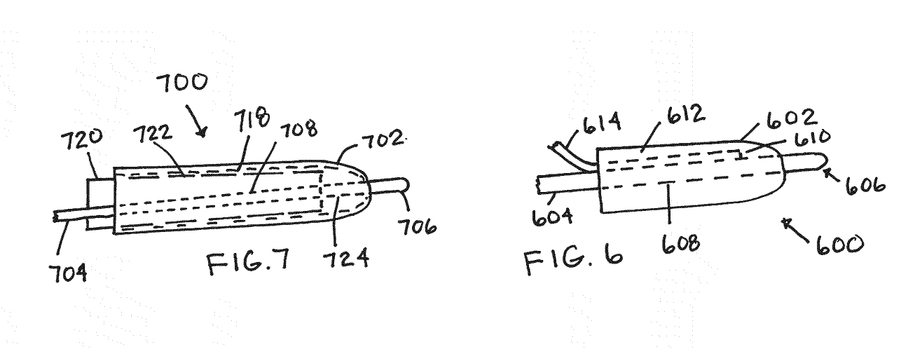

Elastomeric sheath expanded by way of a solid catheter sleeve. A balloon membrane may cover and conform to the catheter shaft to within a few millimeters of the distal tip, leaving a gap between the shaft and an inner (towards the shaft) surface of the balloon membrane. A solid sleeve would then be placed over the catheter shaft. The solid sleeve has an inside diameter (ID) slightly larger than the catheter (outside diameter (OD), and should be able to slide freely along the shaft. The sleeve fits in the gap between the catheter shaft and the elastomeric sheath. After catheter insertion (without the sleeve), the sleeve is inserted and then slid down to the distal end of the catheter, thus displacing the balloon outward, applying a fixed expansion radius. The sleeve can be made in different wall thicknesses in order to allow for selectable amounts of expansion. Additional control over the expansion radius can be provided by a varying diameter sleeve or some fluid (gas or liquid) expansion capacity in the balloon membrane. FIG. 7 displays an embodiment of this system 700 with the elastomeric balloon 702, the proximal end of the catheter 704, the distal end of the catheter 706, the internal (to the balloon) section of the catheter 708 and a sleeve 720 in the internal (to the balloon) section of the catheter 708 from which force from the surface of the sleeve 722 may be delivered against the internal surface 718 of the balloon 702 to create an expanded diameter of the balloon 702. A portion of the catheter 724 is shown that is not covered by the sleeve 720.

Elastomeric sheath expanded by way of self-expanding stent. Plastic or bio-inert metal (e.g., nitinol) self-expanding stents are normally deployed within a catheter guide tube. In this embodiment, the stent and guide tube would be positioned or fit in coaxial alignment around the main catheter shaft, underneath an elastomeric sheath. The catheter-stent-guide-sheath assembly is inserted first, and then the guide tube is withdrawn. Withdrawal of the guide tube allows the stent to expand, displacing the balloon outward.

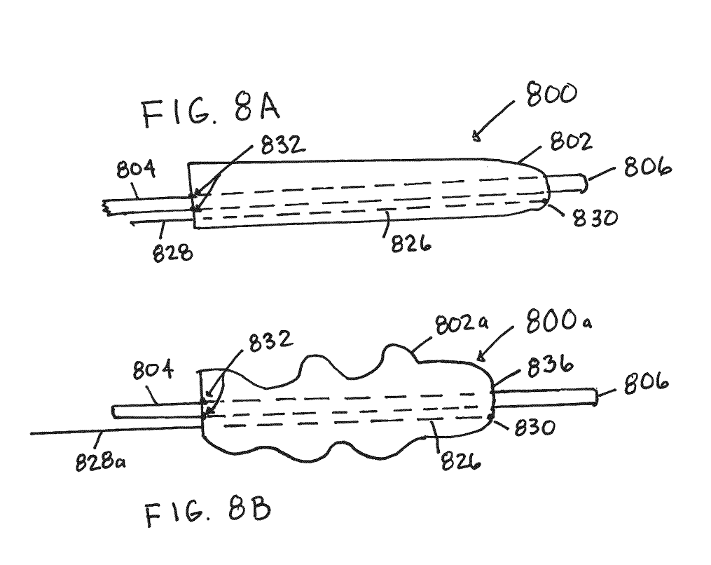

Elastomeric sheath expanded by longitudinal compression. A thin band of elastomeric material (preferably near the tip of the catheter) is compressed longitudinally along the catheter, reducing its length along the catheter and causing the material to effectively expand the band of elastomeric material radially. The compression can be accomplished using a solid catheter sleeve that slides along the catheter shaft or by a retraction component (e.g., pull wire or shaft) that pulls the distal end of the elastomeric material to retract and bunch up to cause effective expansion. Rather than sliding under the elastomer as in the previous embodiment, it pushes on the proximal edge of the elastomer from the proximal side or pulls on the distal edge from the distal side of the elastomeric material. FIG. 8A and FIG. 8B show extended 800a and retracted 800b systems, respectively. Like numbers refer to like elements. A pull wire 828 is shown in FIG. 8A with no significant tension being applied through the pull wire 828 to the securement point 830 on the front end of the elastomeric balloon element 802. There are rear securement or stabilization binding points 832 shown which causes the elastomeric balloon 802 to resist rearward movement along the back of the catheter 804. The portion of the pullwire inside the elastomeric balloon 826 is also shown. In FIG. 8B, the outer portion of the pull wire 828a has been retracted as has the front end 836 of the now distorted and expanded (in regions) balloon 802a.

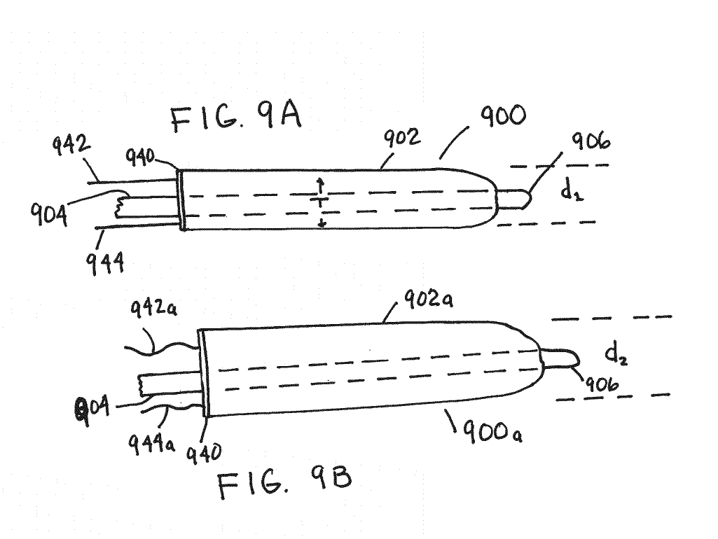

Elastomeric sheath expanded by release of longitudinal expansion. In this variant of the compression sheath, the sheath is stretched laterally along the catheter shaft (and retained in the stretched position during placement), narrowing the elastomeric material radially. The catheter is inserted in this stretched condition, and then the sheath is released from the proximal end (or the distal end), shortening the elastomeric element axially (lengthwise) and thickening it radially. FIGS. 9A and 9B show the system 900 under tension and the system 900a with the tension relaxed. In FIG. 9A, the tension is applied (for example) through pull wires 942 942a, and a cap 940 distributed the force against the balloon 902. Under tension from the pull wires 942 944, the balloon 902 exhibits a first diameter d1, and when the tension from relaxed pull wires 942a 944a has been released, the now expanded balloon 902a exhibits a second diameter d2 which is greater than the original diameter d1. These actions do not significantly impact the catheter 904 or the catheter tip 906 even though the tension T exists across the extended balloon 902.

Infused viscoelastic sealant material. The catheter shaft has a coaxial design in which the intended treatment is infused down the center lumen and out from the distal tip. The outer coaxial lumen is used to infuse a biocompatible thick gel sealant material. Radial ports for egress of the gel sealant are placed a distance of at least several millimeters back from the catheter distal tip and may be distributed radially and longitudinally towards the distal tip or the middle of the catheter. After insertion, a small amount of the gel material is infused, forming a seal around the catheter shaft and displacing some of the tissue at that location. The gel may be sufficiently coherent that it may be retracted into the catheter, back through the ports.

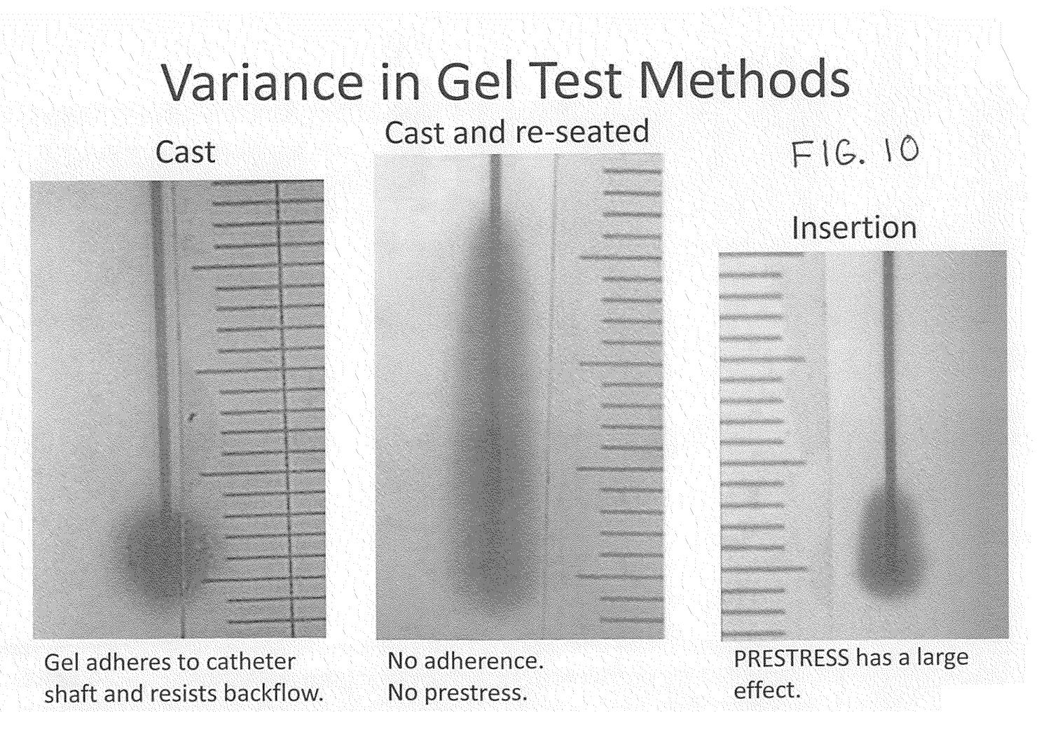

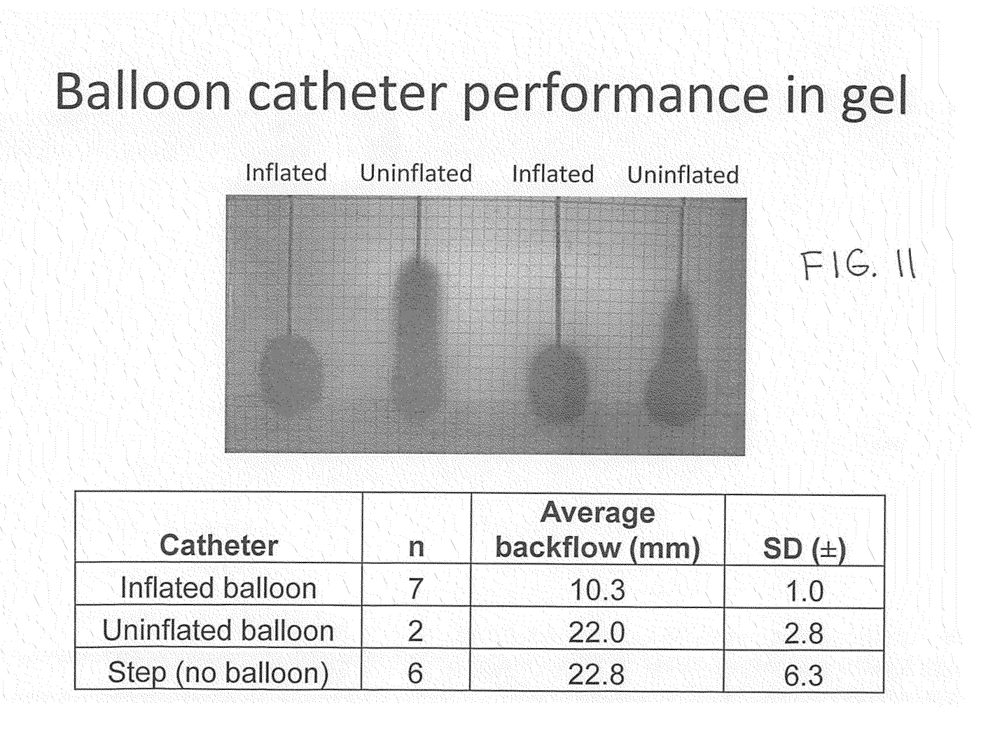

FIG. 10 shows images indicating effects of different applications of pre-stress in gel and their effects on backflow. In the leftmost figure, the catheter was placed in gel during its sol phase. Upon cooling and formation of the gel, the catheter was therefore cast in the gel, and adheres to it. This means that there is maximal pre-stress (adherence, in fact) in the gel-catheter interface, and there is essentially no backflow: the infusion is a spherical ball. In the middle illustration, the catheter was cast in the gel as before, but now the adherence was broken by lifting the catheter up a few millimeters, and then repositioning it to its original place. All the catheter-gel adherence is broken and there is essentially no pre-stress. The adherence is maximal. Finally, in the rightmost figure, the gel is first formed and the catheter inserted into it. This process is complicated physically: if the insertion is rapid, the resistance is of sliding friction, and offers the maximum probability of not tearing the gel or breaking its bonds. If the insertion is slow, the friction may be of the stick-slip kind which would break bonds. The insertion therefore does have a chance of shearing most of the gel away from the catheter, inducing pre-stress, and thus again reducing backflow, albeit somewhat differently from the first case discussed in this paragraph. The resultant reduction in backflow is easily seen in the figure compared with the middle one. The experiments reported in this paragraph were performed by Chris Ross of the Engineering Resources Group in Florida.

FIG. 11 shows an image of balloon catheter performance in gel before and after inflation.

Although specific materials, percentages, and values have been applied in the descriptions of the present technology, alternatives within the generic scope of the disclosure can be selected by those of ordinary skill in the art.

What is claimed:

1. A method for treating an interstitial tissue structure with a flowable medically active or observationally enhancing liquid agent comprising:
providing a delivery cannula comprising at least one catheter or needle having a proximal end and a distal end and an external surface, a lumen assembly extending longitudinally from said proximal to said distal end, and at least one expandable member located on the external surface of the at least one catheter or needle;
inserting the delivery cannula into live tissue, creating a hole in the tissue surrounding the delivery cannula;
establishing contact against an inner surface of the hole with contact pressure from the at least one expandable member, said contact pressure establishing a first level of stress in tissue surrounding the hole in the tissue;
expanding the expandable member to increase the contact pressure and provide a second level of stress in the tissue surrounding the hole in the tissue to a second level of stress that is greater than the first level of stress;
delivering the liquid agent into the tissue through the delivery cannula, the expanded expandable member restricting back flow of the liquid agent towards the proximal ends of the needle or catheter.

2. The method of claim 1 wherein at least one of the contact pressure or the first level of stress are measured.

3. The method of claim 2 wherein at least one of the contact pressure or the first level of stress are measured by a sensor on or in the expandable member.

4. The method of claim 3 wherein at least one of the contact pressure or the first level of stress are measured over at least one time period of at least between one second and at least one minute.

5. The method of claim 3 wherein at least one of the contact pressure or the first level of stress are measured over a continuous time period.

6. The method of claim 4 wherein when a decrease in contact pressure and/or stress in the surrounding tissue is detected, the expandable member is expanded an additional amount to increase measured contact pressure and/or stress in the surrounding tissue.

7. The method of claim 5 wherein when a decrease in contact pressure and/or stress in the surrounding tissue is detected, the expandable member is expanded an additional amount to increase measured contact pressure and/or stress in the surrounding tissue.

8. The method of claim 4 wherein the delivery cannula is positioned at or within a treatment region within the tissue of a patient and the delivery cannula is positioned with the distal end within or proximate to said treatment region.

9. The method of claim 5 wherein the delivery cannula is positioned at or within a treatment region within the tissue of a patient and the delivery cannula is positioned with the distal end within or proximate to said treatment region.

10. The method of claim 6 wherein the delivery cannula is positioned at or within a treatment region within the tissue of a patient and the delivery cannula is positioned with the distal end within or proximate to said treatment region.

11. The method of claim 7 wherein the delivery cannula is positioned at or within a treatment region within the tissue of a patient and the delivery cannula is positioned with the distal end within or proximate to said treatment region.

12. The method of claim 1 wherein in addition to the hole in the tissue, a tear in the tissue extending from the hole is created and a step to resist propagation of the tear in the tissue is performed.

13. The method of claim 6 wherein in addition to the hole in the tissue, a tear in the tissue extending from the hole is created and a step to resist propagation of the tear in the tissue is performed before the expandable member is expanded an additional amount.

14. The method of claim 7 wherein in addition to the hole in the tissue, a tear in the tissue extending from the hole is created and a step to resist propagation of the tear in the tissue is performed before the expandable member is expanded an additional amount.

15. The method of claim 1 wherein the delivery cannula comprises a tubular member having a proximal end and a distal end; said tubular member having a central lumen between said proximal and distal ends; and an infusion tube positioned in said central lumen and extending beyond said proximal and distal ends; said cannula having an plurality of outer surface segments of varying diameter and length; wherein the diameter of each said outer surface segment is substantially uniform along the length of the segment; wherein the relative diameters of said outer surface segments decreases stepwise from the proximal end to the distal end; and wherein the length and diameter of said outer surface segments are selected to reduce reflux during convection-enhanced delivery of substances into tissue in the brain.

16. The method of claim 15 wherein transitions between segments comprise a wall between cannula surfaces of the delivery cannula parallel to the central lumen and wall surface approximately perpendicular to the central lumen, wherein at least some intersections between the cannula surfaces and the wall surfaces are beveled or rounded.

17. A medical device for controlling flow of delivered flowable material within a patient comprising:
e) a cannula having a proximal end and a distal end, and a delivery port towards the distal end;
f) a catheter carrying the cannula, the catheter having an exterior surface and an interior surface;
g) the catheter having a diameter, a distal end and a proximal end, the distal end having an exit port from which the cannula extends or may be extended; and
h) a balloon-element secured to the exterior surface of the catheter between the proximal end and the distal end such that when the balloon-element is expanded, an effectively larger diameter is created about the catheter which can increase force between the secured balloon-element and tissue when the catheter resides within tissue.

18. The device of claim 1 wherein a source of a fluid selected from the group consisting of gas, liquid and gel is associated with an interior volume between the balloon-element and the catheter.

19. The device of claim 18 wherein the balloon element is secured at a proximal end of the balloon-element and a distal end of the balloon element to the exterior surface of the catheter.
